(19) United States
(12) Patent Application Publication
(10) Pub. No.: US 2023/0386623 A1
(43) Pub. Date: Nov. 30, 2023

(54) DRUG AND DIAGNOSIS CONTRAINDICATION IDENTIFICATION USING PATIENT RECORDS AND LAB TEST RESULTS

(71) Applicants: Raymond Anthony Godwin, Jr., Marietta, GA (US); Scott Vander Wielen, Smyrna, GA (US)

(72) Inventors: Raymond Anthony Godwin, Jr., Marietta, GA (US); Scott Vander Wielen, Smyrna, GA (US)

(21) Appl. No.: 18/193,779

(22) Filed: Mar. 31, 2023

Related U.S. Application Data
(60) Provisional application No. 63/347,137, filed on May 31, 2022.

(51) Int. Cl. G16H 10/40; G16H 80/00; G16H 15/00; G16H 10/60

(52) U.S. Cl. CPC G16H 10/40; G16H 80/00; G16H 15/00; G16H 10/60

(57) ABSTRACT

The present disclosure describes a drug and diagnosis contraindication identification platform. The platform may receive a fluid sample from a patient. The fluid sample may be processed to determine one or more substances in the patient's body. Results may be stored in a laboratory information system. The platform may obtain input data from one or more sources. The platform may also receive, as input, a drug that a healthcare worker is considering prescribing or otherwise recommending to the patient. The platform may cross-reference the proposed drug with the one or more substances in the patient's body and/or one or more patient diagnoses from the medical record to determine contraindications. The platform may present the compiled information to the health care professional for consideration prior to prescribing or otherwise recommending the drug to the patient. In some embodiments, the information may be compiled and presented in real-time or substantially in real-time.

### RELATED APPLICATION

Under provisions of 35 U.S.C. § 119(e), the Applicant claims benefit of U.S. Provisional Application No. 63/347,137 filed on May 31, 2022, and having inventors in common, which is incorporated herein by reference in its entirety.

It is intended that the referenced application may be applicable to the concepts and embodiments disclosed herein, even if such concepts and embodiments are disclosed in the referenced application with different limitations and configurations and described using different examples and terminology.

### FIELD OF DISCLOSURE

The present disclosure generally relates to systems for detecting drug interactions.

### BACKGROUND

When a doctor or other health care professional is prescribing or recommending a drug to a patient, they will typically review the patient's medical history to determine if there are any reasons the patient should not take the drug prior to prescribing or recommending. The reasons may include interactions with one or more other drugs the patient is currently taking, a diagnosis that would indicate against taking the drug (e.g., an allergy to the drug or one of its components, a condition that the drug may exacerbate, etc.).

Typically there are databases and websites which provide information regarding contraindications for particular drugs. However, while these websites are complete, they rely on the health care professional having a complete and accurate medical history. However, patients often fail to disclose all substances in their system. In some cases, the patient may not realize that a particular substance, such as a dietary supplement or over-the-counter medication should be disclosed. In other situations, a patient may be nervous or embarrassed about disclosing a drug in their system (e.g., illicit drugs). In still other situations, a patient may not be taking one or more drugs prescribed to them. In still other cases, a patient may have a condition which they are not aware of (e.g., high blood sugar, abnormal hormone levels, early-stage pregnancy, etc.).

Accordingly, there is a need for a platform that allows a healthcare professional to accurately assess the substances present in a patient's system and provide any contra-indications to a particular drug based on the actual assessment, rather than a potentially inaccurate medical chart.

### BRIEF OVERVIEW

This brief overview is provided to introduce a selection of concepts in a simplified form that are further described below in the Detailed Description. This brief overview is not intended to identify key features or essential features of the claimed subject matter. Nor is this brief overview intended to be used to limit the claimed subject matter's scope.

The present disclosure describes a drug and diagnosis contraindication identification platform. The platform may receive a bodily fluid sample (e.g., a blood, urine, and/or saliva sample) from a patient. The bodily fluid sample may be processed by a laboratory in a variety of way to determine one or more substances (e.g., drugs, dietary supplements, etc.) in the patient's body. Results of the test may be stored in a laboratory information system. The platform may obtain input data from one or more sources, including a patient medical record and/or the laboratory information system. The platform may also receive, as input, a drug that a healthcare worker is considering prescribing or otherwise recommending to the patient. The platform may cross-reference the proposed drug with the one or more substances in the patient's body and/or one or more patient diagnoses from the medical record to determine any contraindications. For example, the platform may receive contraindication and or drug-drug interaction data from the National Institute for Health (NIH), the US Food and Drug Administration (FDA) (e.g., via the OpenFDA API), the World Health Organization (WHO), and/or any other trusted medical authority. Accordingly, the platform may compile a list of contraindications and/or drug-drug interactions specific to the patient. The platform may present the compiled information to the health care professional for consideration prior to prescribing or otherwise recommending the drug to the patient. In some embodiments, the information may be compiled and presented in real-time or substantially in real-time.

In some embodiments, the platform may be a system, method, and/or computer program product that receives, from a health care provider, an order comprising patient identification information and an indication of a proposed drug to be administered to the patient. The platform may gather patient information based on the patient identification information, the patient information including a diagnostic history and a list of prescribed medications. The platform tests a bodily fluid sample from a patient, the bodily fluid sample test determining one or more of: one or more substances present within the patient, or one or more patient conditions. The platform may determine one or more contraindications associated with the proposed drug. The one or more contraindications being based on one or more of: a diagnosis present in the diagnostic history, one of the one or more substances present within the patient, or a condition determined by the bodily fluid test. Finally, the platform may provide a report to a health care provider, the report including the one or more determined contraindications.

Both the foregoing brief overview and the following detailed description provide examples and are explanatory only. Accordingly, the foregoing brief overview and the following detailed description should not be considered to be restrictive. Further, features or variations may be provided in addition to those set forth herein. For example, embodiments may be directed to various feature combinations and sub-combinations described in the detailed description.

### BRIEF DESCRIPTION OF THE DRAWINGS

The accompanying drawings, which are incorporated in and constitute a part of this disclosure, illustrate various embodiments of the present disclosure. The drawings contain representations of various trademarks and copyrights owned by the Applicant. In addition, the drawings may contain other marks owned by third parties and are being used for illustrative purposes only. All rights to various trademarks and copyrights represented herein, except those belonging to their respective owners, are vested in and the property of the Applicant. The Applicant retains and reserves all rights in its trademarks and copyrights included herein, and grants permission to reproduce the material only in connection with reproduction of the granted patent and for no other purpose.

Furthermore, the drawings may contain text or captions that may explain certain embodiments of the present disclosure. This text is included for illustrative, non-limiting, explanatory purposes of certain embodiments detailed in the present disclosure. In the drawings:

FIG. 1 illustrates a block diagram of an operating environment consistent with the present disclosure;

FIG. 2 is a flow chart of a method for providing drug and diagnosis contraindication identification using patient records and lab test results; and

FIG. 3 is a block diagram of a system including a computing device for performing the method of FIG. 2.

### DETAILED DESCRIPTION

As a preliminary matter, it will readily be understood by one having ordinary skill in the relevant art that the present disclosure has broad utility and application. As should be understood, any embodiment may incorporate only one or a plurality of the above-disclosed aspects of the disclosure and may further incorporate only one or a plurality of the above-disclosed features. Furthermore, any embodiment discussed and identified as being “preferred” is considered to be part of a best mode contemplated for carrying out the embodiments of the present disclosure. Other embodiments also may be discussed for additional illustrative purposes in providing a full and enabling disclosure. Moreover, many embodiments, such as adaptations, variations, modifications, and equivalent arrangements, will be implicitly disclosed by the embodiments described herein and fall within the scope of the present disclosure.

Accordingly, while embodiments are described herein in detail in relation to one or more embodiments, it is to be understood that this disclosure is illustrative and exemplary of the present disclosure and are made merely for the purposes of providing a full and enabling disclosure. The detailed disclosure herein of one or more embodiments is not intended, nor is to be construed, to limit the scope of patent protection afforded in any claim of a patent issuing here from, which scope is to be defined by the claims and the equivalents thereof. It is not intended that the scope of patent protection be defined by reading into any claim a limitation found herein that does not explicitly appear in the claim itself.

Thus, for example, any sequence(s) and/or temporal order of steps of various processes or methods that are described herein are illustrative and not restrictive. Accordingly, it should be understood that, although steps of various processes or methods may be shown and described as being in a sequence or temporal order, the steps of any such processes or methods are not limited to being carried out in any particular sequence or order, absent an indication otherwise. Indeed, the steps in such processes or methods generally may be carried out in various different sequences and orders while still falling within the scope of the present invention. Accordingly, it is intended that the scope of patent protection is to be defined by the issued claim(s) rather than the description set forth herein.

Additionally, it is important to note that each term used herein refers to that which an ordinary artisan would understand such term to mean based on the contextual use of such term herein. To the extent that the meaning of a term used herein—as understood by the ordinary artisan based on the contextual use of such term—differs in any way from any particular dictionary definition of such term, it is intended that the meaning of the term as understood by the ordinary artisan should prevail.

Regarding applicability of 35 U.S.C. § 112, ¶6, no claim element is intended to be read in accordance with this statutory provision unless the explicit phrase “means for” or “step for” is actually used in such claim element, whereupon this statutory provision is intended to apply in the interpretation of such claim element.

Furthermore, it is important to note that, as used herein, “a” and “an” each generally denotes “at least one,” but does not exclude a plurality unless the contextual use dictates otherwise. When used herein to join a list of items, “or” denotes “at least one of the items,” but does not exclude a plurality of items of the list. Finally, when used herein to join a list of items, “and” denotes “all of the items of the list.”

The following detailed description refers to the accompanying drawings. Wherever possible, the same reference numbers are used in the drawings and the following description to refer to the same or similar elements. While many embodiments of the disclosure may be described, modifications, adaptations, and other implementations are possible. For example, substitutions, additions, or modifications may be made to the elements illustrated in the drawings, and the methods described herein may be modified by substituting, reordering, or adding stages to the disclosed methods. Accordingly, the following detailed description does not limit the disclosure. Instead, the proper scope of the disclosure is defined by the appended claims. The present disclosure contains headers. It should be understood that these headers are used as references and are not to be construed as limiting upon the subjected matter disclosed under the header.

The present disclosure includes many aspects and features. Moreover, while many aspects and features relate to, and are described in, the context of a drug and diagnosis contraindication identification platform, embodiments of the present disclosure are not limited to use only in this context.

### I. Platform Overview

This overview is provided to introduce a selection of concepts in a simplified form that are further described below. This overview is not intended to identify key features or essential features of the claimed subject matter. Nor is this overview intended to be used to limit the claimed subject matter's scope.

A drug and diagnosis contraindication identification platform may receive a bodily fluid sample (e.g., a blood, urine, and/or saliva sample) from a patient. The bodily fluid sample may be processed by a laboratory in a variety of way to determine one or more substances (e.g., drugs, dietary supplements, etc.) in the patient's body. Results of the test may be stored in a laboratory information system. The platform may obtain input data from one or more sources, including a patient medical record and/or the laboratory information system. The platform may also receive, as input, a drug that a healthcare worker is considering prescribing or otherwise recommending to the patient. The platform may cross-reference the proposed drug with the one or more substances in the patient's body and/or one or more patient diagnoses from the medical record to determine any contraindications. For example, the platform may receive contraindication and or drug-drug interaction data from the National Institute for Health (NIH), the US Food and Drug Administration (FDA) (e.g., via the OpenFDA API), the World Health Organization (WHO), and/or any other trusted medical authority. Accordingly, the platform may compile a list of contraindications and/or drug-drug interactions specific to the patient. The platform may present the compiled information to the health care professional for consideration prior to prescribing or otherwise recommending the drug to the patient. In some embodiments, the information may be compiled and presented in real-time or substantially in real-time.

Embodiments of the present disclosure may comprise methods, systems, and a computer readable medium comprising, but not limited to, at least one of the following:
A. A Medical Record Management Module;
B. A Bodily Fluid Testing System;
C. A Laboratory Information System;
D. A Data Collection and Comparison Module;

Details with regards to each module is provided below. Although modules are disclosed with specific functionality, it should be understood that functionality may be shared between modules, with some functions split between modules, while other functions duplicated by the modules. Furthermore, the name of the module should not be construed as limiting upon the functionality of the module. Moreover, each component disclosed within each module can be considered independently without the context of the other components within the same module or different modules. Each component may contain language defined in other portions of this specifications. Each component disclosed for one module may be mixed with the functionality of another module. In the present disclosure, each component can be claimed on its own and/or interchangeably with other components of other modules.

The following depicts an example of a method of a plurality of methods that may be performed by at least one of the aforementioned modules, or components thereof. Various hardware components may be used at the various stages of operations disclosed with reference to each module. For example, although methods may be described to be performed by a single computing device, it should be understood that, in some embodiments, different operations may be performed by different networked elements in operative communication with the computing device. For example, at least one computing device 300 may be employed in the performance of some or all of the stages disclosed with regard to the methods. Similarly, an apparatus may be employed in the performance of some or all of the stages of the methods. As such, the apparatus may comprise at least those architectural components as found in computing device 300.

Furthermore, although the stages of the following example method are disclosed in a particular order, it should be understood that the order is disclosed for illustrative purposes only. Stages may be combined, separated, reordered, and various intermediary stages may exist. Accordingly, it should be understood that the various stages, in various embodiments, may be performed in arrangements that differ from the ones claimed below. Moreover, various stages may be added or removed without altering or deterring from the fundamental scope of the depicted methods and systems disclosed herein.

Consistent with embodiments of the present disclosure, a method may be performed by at least one of the modules disclosed herein. The method may be embodied as, for example, but not limited to, computer instructions, which when executed, perform the method. The method may comprise the following stages:
Gathering patient information stored in a patient medical record;
Testing a bodily fluid sample from a patient, the bodily fluid sample test determining one or more of:
   one or more substance present within the patient, or
   one or more patient conditions;
Receiving, form a health care provider, a proposed drug associated with the patient;
Determining one or more contra-indications the proposed drug, the one or more contra-indications being based on one or more of
   a diagnosis present in the medical chart,
   one of the one or more substances present within the patient, or
   a condition determined by the bodily fluid test; Providing the one or more determined contra-indications to the health care provider.

Although the aforementioned method has been described to be performed by the platform 100, it should be understood that computing device 300 may be used to perform the various stages of the method. Furthermore, in some embodiments, different operations may be performed by different networked elements in operative communication with computing device 300. For example, a plurality of computing devices may be employed in the performance of some or all of the stages in the aforementioned method. Moreover, a plurality of computing devices may be configured much like a single computing device 300. Similarly, an apparatus may be employed in the performance of some or all stages in the method. The apparatus may also be configured much like computing device 300.

Both the foregoing overview and the following detailed description provide examples and are explanatory only. Accordingly, the foregoing overview and the following detailed description should not be considered to be restrictive. Further, features or variations may be provided in addition to those set forth herein. For example, embodiments may be directed to various feature combinations and sub-combinations described in the detailed description.

### II. Platform Configuration

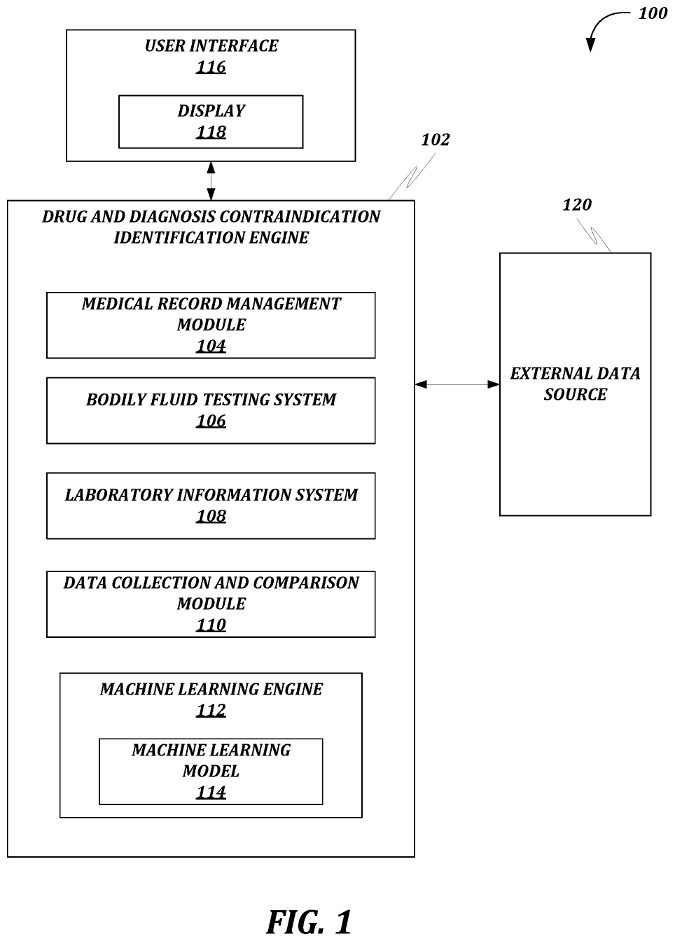

FIG. 1 illustrates one possible operating environment through which a platform consistent with embodiments of the present disclosure may be provided. By way of non-limiting example, a drug and diagnosis contraindication identification platform 100 may be hosted on, for example, a cloud computing service. In some embodiments, the platform 100 may be hosted on a computing device 300. A user may access platform 100 through a software application and/or hardware device. The software application may be embodied as, for example, but not be limited to, a website, a web application, a desktop application, and a mobile application compatible with the computing device 300.

In embodiments, the platform 100 may include a drug and diagnosis contraindication identification engine 102, a user interface 116, one or more external data sources 120, and various components thereof. In one or more embodiments, the platform 100 may include more or fewer components than the components illustrated in FIG. 1. The components illustrated in FIG. 1 may be local to or remote from each other.

In one or more embodiments, the user interface 116 refers to hardware and/or software configured to facilitate communications between a user and the drug and diagnosis contraindication identification platform 100. The user interface 116 may be used by a user who accesses an interface (e.g., a dashboard interface) for work and/or personal activities. The user interface 116 may be associated with one or more devices for presenting visual media, such as a display 118, including a monitor, a television, a projector, and/or the like. User interface 116 renders user interface elements and receives input via user interface elements. Examples of interfaces include a graphical user interface (GUI), a command line interface (CLI), a haptic interface, and a voice command interface. Examples of user interface elements include checkboxes, radio buttons, menus, dropdown lists, list boxes, buttons, toggles, text fields, date and time selectors, command lines, sliders, pages, and forms.

In an embodiment, different components of the user interface 116 are specified in different languages. The behavior of user interface elements may be specified in a dynamic programming language, such as JavaScript. The content of user interface elements may be specified in a markup language, such as hypertext markup language (HTML) or XML User Interface Language (XUL). The layout of user interface elements may be specified in a style sheet language, such as Cascading Style Sheets (CSS). Alternatively, the user interface 116 is specified in one or more other languages, such as Java, C, or C++.

In embodiments, the drug and diagnosis contraindication identification engine 102 may include a software and/or hardware platform comprised of a distributed set of computing elements, including, but not limited to: A network feature extraction module 104 may refer to hardware and/or software configured to perform operations described herein (including such operations as may be incorporated by reference) for extracting one or more network features from a network associated with an entity to be classified.

A. A Medical Record Management Module

A medical record management module 104 may refer to hardware and/or software configured to perform operations described herein (including such operations as may be incorporated by reference) for extracting patient data associated with a particular patient from a medical record system, such as an electronic medical record system. The medical record management module 104 may be configured to interface with an electronic medical record (EMR) system, and may be configured to use the various security protocols enforced by such an EMR system. In particular, the medical record management module 104 may use one or more security protocols compliant with the Health Insurance Portability and Accountability Act (HIPAA). Additionally or alternatively, the medical record management module 104 may comply with standards set forth by the World Health Organization (WHO), the Personal Information Protection and Electronic Documents Act (PIPEDA) of Canada, the General Data Protection Regulations (GDPR) of the European Union, and/or any other data privacy regulations.

B. A Bodily Fluid Testing System

A bodily fluid testing system 106 may refer to hardware and/or software configured to perform operations described herein (including such operations as may be incorporated by reference) for testing a bodily fluid sample (e.g., a blood sample) from a patient. For example, the bodily fluid testing system may include equipment configured to analyze the bodily fluid sample to determine one or more substances present in the bodily fluid sample. For example, the equipment may determine one or more substance (e.g., drugs, dietary supplements, etc.) present in a blood sample drawn from the patient. As non-limiting examples, the equipment may include liquid chromatography mass spectrometry (LC-MS/MS) instrumentation, gas chromatography mass spectrometry (GC-MS/MS) instrumentation, and/or any other equipment or instrumentation capable of separating and identifying substances dispersed in the bodily fluid sample.

In some embodiments, the bodily fluid testing system 106 may be configured for testing one or more of the following bodily fluids: blood, urine, cerebrospinal fluid, saliva, sweat, and/or other bodily fluids for presence of one or more compounds in the fluid. The bodily fluid testing system 106 may determine one or more compounds present within the bodily fluid sample. For example, the compounds may include one or more of a particular substance taken (e.g., ingested, inhaled, injected, etc.) by the patient, a metabolite or antigen associated with a particular compound, and/or any other indication that the compound is present in the patient.

C. A Laboratory Information System

A laboratory information system 108 may refer to hardware and/or software configured to perform operations described herein (including such operations as may be incorporated by reference) for interpreting and/or storing the results of the bodily fluid testing system 106. In particular, the laboratory information system 108 may cause the bodily fluid testing system 106 to perform one or more tests or assays for determining presence of one or more compounds within a bodily fluid. Upon completion of the tests or assays by the bodily fluid testing system 106, the laboratory information system 108 may cause results to be stored to an electronic medical record using the medical record management module 104. In some embodiments, the laboratory information system 108 may cause the one or more tests or assay to be completed based on an order received from a device associated with a physician or other medical professional.

In some embodiments, the laboratory information system 108 may further interpret results of the one or more tests or assays using the data collection and comparison module 110. Interpreting the results may include interpreting results of each individual test or assay on its own, interpreting the results of a particular test or assay based at least in part on one or more other tests or assays, an/or interpreting the results of a particular test or assay based at least in part on patient history data (e.g., from the EMR and/or the data collection and comparison module 110).

In some embodiments, interpreting results of the one or more tests or assays may include making a determination regarding whether or not a particular compound is present in the patient. In some embodiments, interpreting the results may include providing a confidence level associated with such a determination.

D. A Data Collection and Comparison Module

A data collection and comparison module 110 may refer to hardware and/or software configured to perform operations described herein (including such operations as may be incorporated by reference) for compiling data (e.g., from the laboratory information system 106 and the medical record management module 104 In embodiments the data collection and comparison module 108 may include hardware and/or software configured to determine contraindications and/or drug-drug interactions for a proposed drug based on the compiled data indicating current patient drug and diagnosis information.

In an embodiment, one or more components of the drug and diagnosis contraindication identification engine 102 use an artificial intelligence, such as a machine learning engine 112. For example, the machine learning engine 112 may be used to determine one or more substances in a patient's bodily fluid sample (e.g., by the bodily fluid testing system 106) and/or to determine contraindications and/or drug-drug interactions for a proposed drug (e.g., by the data collection and comparison module 110). Machine learning includes various techniques in the field of artificial intelligence that deal with computer-implemented, user-independent processes for solving problems that have variable inputs.

In some embodiments, the machine learning engine 112 trains a machine learning model 114 to perform one or more operations. Training a machine learning model 114 uses training data to generate a function that, given one or more inputs to the machine learning model 114, computes a corresponding output. The output may correspond to a prediction based on prior machine learning. In an embodiment, the output includes a label, classification, and/or categorization assigned to the provided input(s). The machine learning model 114 corresponds to a learned model for performing the desired operation(s) (e.g., labeling, classifying, and/or categorizing inputs). The drug and diagnosis contraindication identification engine 102 may use multiple machine learning engines 112 and/or multiple machine learning models 114 for different purposes.

In an embodiment, the machine learning engine 112 may use supervised learning, semi-supervised learning, unsupervised learning, reinforcement learning, and/or another training method or combination thereof. In supervised learning, labeled training data includes input/output pairs in which each input is labeled with a desired output (e.g., a label, classification, and/or categorization), also referred to as a supervisory signal. In semi-supervised learning, some inputs are associated with supervisory signals and other inputs are not associated with supervisory signals. In unsupervised learning, the training data does not include supervisory signals. Reinforcement learning uses a feedback system in which the machine learning engine 112 receives positive and/or negative reinforcement in the process of attempting to solve a particular problem (e.g., to optimize performance in a particular scenario, according to one or more predefined performance criteria). One example of a network for use in reinforcement learning is a recurrent neural network, which may include a backpropagation or feedback pathway to correct or improve the response of the network.

In an embodiment, a machine learning engine 112 may use many different techniques to label, classify, and/or categorize inputs. A machine learning engine 112 may transform inputs (e.g., the patient data, bodily fluid test results, etc.) into feature vectors that describe one or more properties (“features”) of the inputs. The machine learning engine 112 may label, classify, and/or categorize the inputs based on the feature vectors. Alternatively or additionally, a machine learning engine 112 may use clustering (also referred to as cluster analysis) to identify commonalities in the inputs. The machine learning engine 112 may group (i.e., cluster) the inputs based on those commonalities. The machine learning engine 112 may use hierarchical clustering, k-means clustering, and/or another clustering method or combination thereof. In an embodiment, a machine learning engine 112 includes an artificial neural network. An artificial neural network includes multiple nodes (also referred to as artificial neurons) and edges between nodes. Edges may be associated with corresponding weights that represent the strengths of connections between nodes, which the machine learning engine 112 adjusts as machine learning proceeds. Alternatively or additionally, a machine learning engine 112 may include a support vector machine. A support vector machine represents inputs as vectors. The machine learning engine 112 may label, classify, and/or categorizes inputs based on the vectors. Alternatively or additionally, the machine learning engine 112 may use a naïve Bayes classifier to label, classify, and/or categorize inputs. Alternatively or additionally, given a particular input, a machine learning model may apply a decision tree to predict an output for the given input. Alternatively or additionally, a machine learning engine 112 may apply fuzzy logic in situations where labeling, classifying, and/or categorizing an input among a fixed set of mutually exclusive options is impossible or impractical. The aforementioned machine learning model 114 and techniques are discussed for exemplary purposes only and should not be construed as limiting one or more embodiments.

In an embodiment, as a machine learning engine 112 applies different inputs to a machine learning model 114, the corresponding outputs are not always accurate. As an example, the machine learning engine 112 may use supervised learning to train a machine learning model 114. After training the machine learning model 114, if a subsequent input is identical to an input that was included in labeled training data and the output is identical to the supervisory signal in the training data, then output is certain to be accurate. If an input is different from inputs that were included in labeled training data, then the machine learning engine 112 may generate a corresponding output that is inaccurate or of uncertain accuracy. In addition to producing a particular output for a given input, the machine learning engine 112 may be configured to produce an indicator representing a confidence (or lack thereof) in the accuracy of the output. A confidence indicator may include a numeric score, a Boolean value, and/or any other kind of indicator that corresponds to a confidence (or lack thereof) in the accuracy of the output.

In an embodiment, the drug and diagnosis contraindication identification engine 102 is configured to receive data from one or more external data sources 120. An external data source 120 refers to hardware and/or software operating independent of the drug and diagnosis contraindication identification engine 102. For example, the hardware and/or software of the external data source 120 may be under control of a different entity (e.g., a different company or other kind of organization) than an entity that controls the entity classification and data risk assessment engine. An external data source 120 may include, for example, one or more sources of contraindication and/or drug-drug interaction data, such as those operated by the US Food and Drug Administration (FDA), the National Institute for Health (NIH), the World Health Organization (WHO), and/or any other national or regional healthcare organization established around the world.

In an embodiment, the drug and diagnosis contraindication identification engine 102 is configured to retrieve data from an external data source 120 by ‘pulling’ the data via an application programming interface (API) of the external data source 120, using user credentials that a user has provided for that particular external data source 120. As a particular example, the FDA uses the OpenFDA API to allow users to retrieve contraindication and/or drug-drug interaction data. Alternatively or additionally, an external data source 120 may be configured to ‘push’ data to the drug and diagnosis contraindication identification engine 102 via an API, using an access key, password, and/or other kind of credential that a user has supplied to the external data source 120. The drug and diagnosis contraindication identification engine 102 may be configured to receive data from an external data source 120 in many different ways.

### III. Platform Operation

Embodiments of the present disclosure provide a hardware and software platform operative by a set of methods and computer-readable media comprising instructions configured to operate the aforementioned modules and computing elements in accordance with the methods. The following depicts an example of at least one method of a plurality of methods that may be performed by at least one of the aforementioned modules. Various hardware components may be used at the various stages of operations disclosed with reference to each module.

For example, although methods may be described to be performed by a single computing device, it should be understood that, in some embodiments, different operations may be performed by different networked elements in operative communication with the computing device. For example, at least one computing device 300 may be employed in the performance of some or all of the stages disclosed with regard to the methods. Similarly, an apparatus may be employed in the performance of some or all of the stages of the methods. As such, the apparatus may comprise at least those architectural components as found in computing device 300.

Furthermore, although the stages of the following example method are disclosed in a particular order, it should be understood that the order is disclosed for illustrative purposes only. Stages may be combined, separated, reordered, and various intermediary stages may exist. Accordingly, it should be understood that the various stages, in various embodiments, may be performed in arrangements that differ from the ones claimed below. Moreover, various stages may be added or removed from the without altering or deterring from the fundamental scope of the depicted methods and systems disclosed herein.

A. Method of Identifying Drug and Diagnosis Contraindications

Consistent with embodiments of the present disclosure, a method may be performed by at least one of the aforementioned modules. The method may be embodied as, for example, but not limited to, computer instructions, which when executed, perform the method.

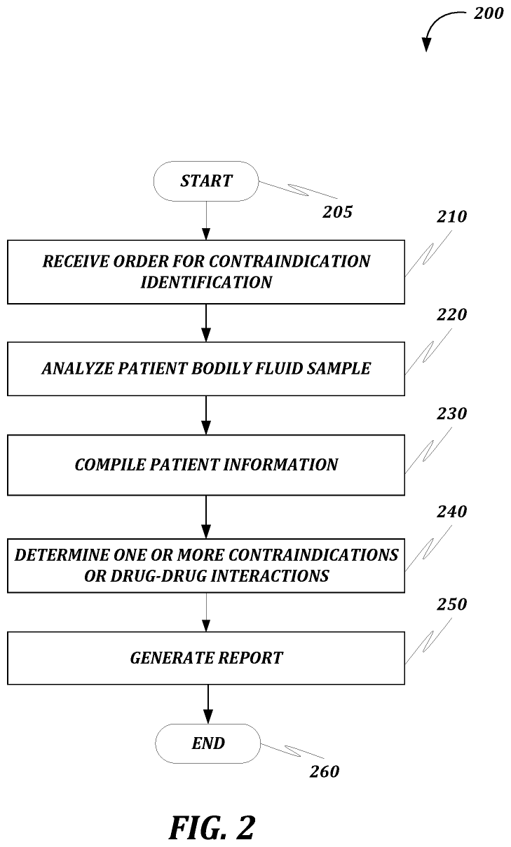

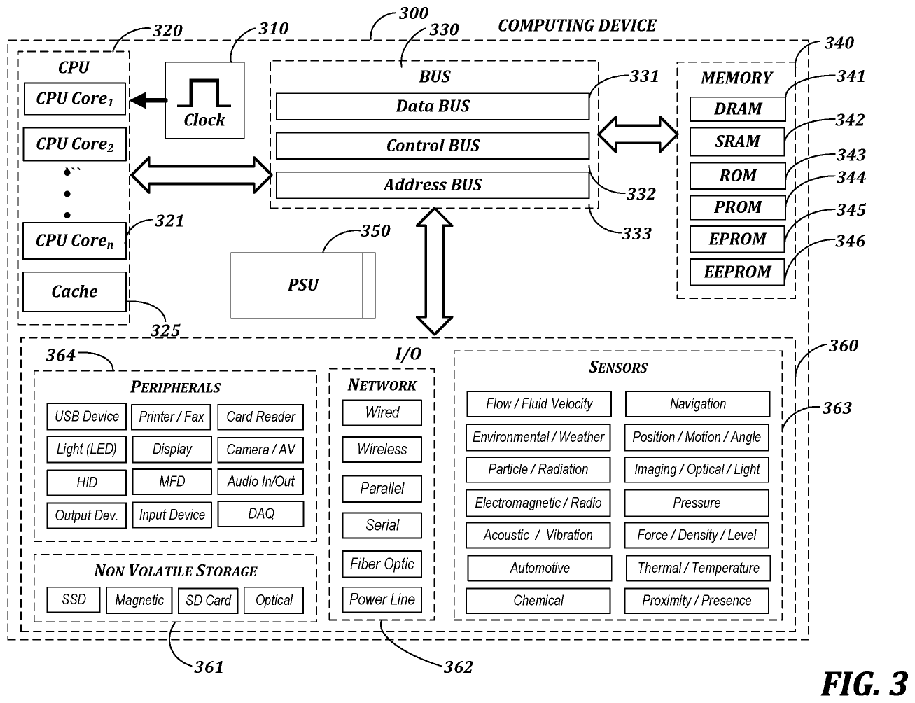

FIG. 2 is a flow chart setting forth the general stages involved in a method 200 consistent with an embodiment of the disclosure for using the drug and diagnosis contraindication identification platform 100 to. Method 200 may be implemented using a computing device 300 or any other component associated with platform 100 as described in more detail below with respect to FIG. 3. For illustrative purposes alone, computing device 300 is described as one potential actor in the follow stages.

Method 200 may begin at starting block 205 and proceed to stage 210 where computing device 300 may receive an order for a drug and diagnosis contraindication identification. In some embodiments, the order may include order information. For example, the order information may comprise patient demographic information, patient identification information, proposed drug information and/or payer information. In some embodiments, the order information may include one or more diagnostic codes associated with the patient and/or patient prescription information.

The patient demographic information may include background information about the patient useful for performing an analysis of a bodily fluid sample. As an example, the demographic information may include generalized or specific patient demographic information including (but not limited to) age, gender, ethnicity, and/or other information useful for analyzing the bodily fluid sample.

The patient identification information may include specific information that identifies a particular patient associated with a bodily fluid sample to be analyzed. For example, the patient identification information may include (but need not be limited to) one or more of a patient name, a patient birth date, a patient identification number (e.g., a hospital admission or case number, a social security number, etc.), and/or other information that identifies the patient. In some embodiments, the patient identification information may include information which may be used to unambiguously identify the particular patient within an EMR system and to obtain an EMR associated with the particular patient.

The proposed drug information may include an indication of one or more drugs that a physician or other medical professional is interested in prescribing or otherwise administering to the particular patient.

The payer information may include information associated with an insurance plan or other entity responsible for providing payment for the testing performed by the system.

From stage 210, where computing device 300 receives the order, method 200 may advance to stage 220 where computing device 300 may analyze a bodily fluid sample associated with the patient. For example, the computing device 300 may perform one or more analyses to determine one or more substances (e.g., drugs, dietary supplements, etc.) present in the bodily fluid sample As particular examples, the bodily fluid sample may comprise a blood sample, a urine sample, a saliva sample, a cerebrospinal fluid sample, a sweat sample, and/or any other bodily fluid sample procured from a patient. The computing device 300 may perform analyses such as (but not limited to) liquid chromatography mass spectrometry and/or gas chromatography mass spectrometry analyses on the sample to determine one or more substances in the sample. In embodiments, the analyses performed may be determined by various factors, including the type of fluid sample obtained, patient history information, patient admission formation proposed drug information, patient demographic information, and/or other factors that may affect the level of detail required in determining substance in the fluid sample. In embodiments, data associated with the one or more analyses may be stored to a data store for use by the computing system 300.

Method 200 may continue to stage 230 where computing device 300 may compile patient information. In embodiments, compiling patient information may include retrieving the results of the bodily fluid test performed in stage 220 to determine one or more substances in the patient's blood. In some embodiment, compiling patient information may include determining patient diagnostic information, patient demographic information, and/or the like. For example, compiling patient information may include the computing extracting patient information from the order information received in stage 210. As another example, compiling patient information may include the computing device retrieving patient information from an electronic medical record system (e.g., based on patient identification information extracted from the order information received in stage 210).

In embodiments, the results of the bodily fluid analyses may be interpreted and/or stored using a laboratory information system. In some embodiments, the results for one or more (e.g., each) of the analyses may be interpreted individually, without reference to any outside data. In some embodiments, the results for one or more (e.g., each) of the analyses may be interpreted with reference to at least results of at least one other analysis. In some embodiments, the results for one or more (e.g., each) of the analyses may be interpreted individually, with reference to at least a portion of the compiled patient information (e.g., the patient demographic information, the patient identification information, and/or information from a patient EMR). The test results may indicate one or more substances that are in the patient system related to prescriptions, over-the-counter medications (e.g., acetaminophen, ibuprofen, diphenhydramine, etc.), natural supplements (e.g., coenzyme Q10, vitamin D, vitamin B12, echinacea, etc.), dietary substances (e.g., caffeine, nicotine, alcohol), illicit drugs, and/or any other substance within the patient system. The computing device may determine one or more of many substances in the patient system. The computing device may save a list of determined substances in the patient system to a medical record associated with the patient.

In some embodiments, for each substance determined to be in the patient system, the computing device may further compute a confidence score associated with the presence of the substance. In some embodiments, the list of determined substances may further comprise the confidence level associated with each substance. Additionally or alternatively, the computing device may omit substances from the list if the confidence level is below a threshold amount.

After computing device 300 compiles the patient information in stage 230, method 200 may proceed to stage 240 where computing device 300 may determine whether one or more contraindications and/or drug-drug interactions exist. In particular, the computing device may check for interactions between and among the proposed drug information and the compiled patient information from stage 230. For example, the computing device may query one or more databases using the proposed drug information from the order information received in stage 210 to determine a list of substances that interact with the proposed drug. The computing device 300 may compare the determined list of substances that interact with the proposed drug to the list of substance in the patient system to determine if there is an interaction or contraindication for the proposed drug. Alternatively, the system may transmit one or more database queries to a drug interaction database, with each query comprising (but not limited to) an indication of the proposed drug (e.g., based on the proposed drug information) and an indication of one substance from the list of substances in the patient system. Responsive to each query, the drug interaction database may respond with information indicating whether there is a contraindication for the two substances (e.g., the proposed drug and the substance from the list) being administered concurrently to a patient.

In some embodiments, the one or more contraindications and/or drug-drug interactions may comprise an interaction between the proposed drug and one or more of the substances present in the patient's system (e.g., blood, saliva, cerebrospinal fluid, urine, etc.). Alternatively or additionally, the contraindications and/or drug-drug interactions may comprise one or more contraindications based on diagnostic information and/or patient demographic information and the proposed drug (e.g., the patient has high blood pressure and the drug causes increased blood pressure). In some embodiments, the one or more contraindications and/or drug-drug interactions may comprise an interaction between two substances present in the patient's system. In some embodiments, the computing device may crosswalk different diagnosis code language to successfully complete a query. In embodiments, the one or more databases may include, an NIH database, an FDA database, and/or any other database or trusted repository of medical drug contraindication and/or interaction data.

Once computing device 300 determines existence of one or more contraindications and/or drug-drug interactions in stage 240, method 200 may continue to stage 250 where computing device 300 may generate a report regarding the patient. In embodiments, the report may indicate the contraindications and/or drug-drug interaction information for the drugs and diagnoses either provided in the prescription list or observed in lab tests. Alternatively or additionally, the report may include an indication, for each prescription in the patient's prescription information, whether the patient is compliant with the prescription. The report may be a text-based report, and may be generated in any format readable by a healthcare worker. For example, the report may be created in Adobe Portable Document Format (PDF) such that text, images, and the like may be included and formatted for review across multiple platforms. In embodiments, the report may be provided via any method of transmitting an electronic document, including displaying on a screen, causing the report to be printed, placing the report in the patient's electronic medical record, transmitting the report via email or SMS, and/or the like. In this way, the method may create a report that accurately reflects whether a proposed drug would have any contraindications or drug-drug interactions with substances actually in the patient's system, whether prescribed or otherwise. The report may include information about whether the drug includes any contraindications based on the patient's diagnostic history as well. In some embodiments, the report may also be used to monitor patient compliance with prescribed medications and/or to screen for unprescribed substance being used by the patient. The report may be generated in real time or substantially in real time, allowing the healthcare worker to verify a proposed drug prior to actually prescribing or recommending the drug to the patient.

In some embodiments, where there is an interaction between the proposed drug and a substance in the patient's system, the computing device may cancel a prescription order for the proposed drug. In some embodiments, responsive to a substance in the patient system that is not accounted for in the patient history data, the computing device may alert a physician to the additional substances in the patient's system. Similarly, responsive to the computing device determining that the patient is not taking a medication or substance (e.g., a prescription) indicated in the patient history data, the computing device may similarly alert a physician. In some embodiments, given the patient data and the proposed drug, the computing device may recommend one or more alternative medications that are not contra-indicated and/or do not interact with substances in the patient system. In some embodiments, in response to determining that one or more illicit drugs are present in the patient's system, the computing device may transmit a recommendation to a physician and/or to the patient for substance abuse treatment plans.

Once computing device 300 generates the report in stage 250, method 200 may then end at stage 260.

### IV. Computing Device Architecture

Embodiments of the present disclosure provide a hardware and software platform operative as a distributed system of modules and computing elements.

Platform 100 may be embodied as, for example, but not be limited to, a website, a web application, a desktop application, backend application, and a mobile application compatible with a computing device 300. The computing device 300 may comprise, but not be limited to the following:
Mobile computing device, such as, but is not limited to, a laptop, a tablet, a smartphone, a drone, a wearable, an embedded device, a handheld device, an Arduino, an industrial device, or a remotely operable recording device;
A supercomputer, an exa-scale supercomputer, a mainframe, or a quantum computer;
A minicomputer, wherein the minicomputer computing device comprises, but is not limited to, an IBM AS400/iSeries/System I, A DEC VAX/PDP, a HP3000, a Honeywell-Bull DPS, a Texas Instruments TI-990, or a Wang Laboratories VS Series;
A microcomputer, wherein the microcomputer computing device comprises, but is not limited to, a server, wherein a server may be rack mounted, a workstation, an industrial device, a raspberry pi, a desktop, or an embedded device;
Platform 100 may be hosted on a centralized server or a cloud computing service. Although method 200 has been described to be performed by a computing device 300, it should be understood that, in some embodiments, different operations may be performed by a plurality of the computing devices 300 in operative communication at least one network.

Embodiments of the present disclosure may comprise a system having a central processing unit (CPU) 320, a bus 330, a memory unit 340, a power supply unit (PSU) 350, and one or more Input/Output (I/O) units. The CPU 320 coupled to the memory unit 340 and the plurality of I/O units 360 via the bus 330, all of which are powered by the PSU 350. It should be understood that, in some embodiments, each disclosed unit may actually be a plurality of such units for the purposes of redundancy, high availability, and/or performance. The combination of the presently disclosed units is configured to perform the stages any method disclosed herein.

FIG. 3 is a block diagram of a system including computing device 300. Consistent with an embodiment of the disclosure, the aforementioned CPU 320, the bus 330, the memory unit 340, a PSU 350, and the plurality of I/O units 360 may be implemented in a computing device, such as computing device 300 of FIG. 3. Any suitable combination of hardware, software, or firmware may be used to implement the aforementioned units. For example, the CPU 320, the bus 330, and the memory unit 340 may be implemented with computing device 300 or any of other computing devices 300, in combination with computing device 300. The aforementioned system, device, and components are examples and other systems, devices, and components may comprise the aforementioned CPU 320, the bus 330, the memory unit 340, consistent with embodiments of the disclosure.

At least one computing device 300 may be embodied as any of the computing elements illustrated in all of the attached figures. A computing device 300 does not need to be electronic, nor even have a CPU 320, nor bus 330, nor memory unit 340. The definition of the computing device 300 to a person having ordinary skill in the art is “A device that computes, especially a programmable [usually] electronic machine that performs high-speed mathematical or logical operations or that assembles, stores, correlates, or otherwise processes information.” Any device which processes information qualifies as a computing device 300, especially if the processing is purposeful.

With reference to FIG. 3, a system consistent with an embodiment of the disclosure may include a computing device, such as computing device 300. In a basic configuration, computing device 300 may include at least one clock module 310, at least one CPU 320, at least one bus 330, and at least one memory unit 340, at least one PSU 350, and at least one I/O 360 module, wherein I/O module may be comprised of, but not limited to a non-volatile storage sub-module 361, a communication sub-module 362, a sensors sub-module 363, and a peripherals sub-module 364.

A system consistent with an embodiment of the disclosure the computing device 300 may include the clock module 310 may be known to a person having ordinary skill in the art as a clock generator, which produces clock signals. Clock signal is a particular type of signal that oscillates between a high and a low state and is used like a metronome to coordinate actions of digital circuits. Most integrated circuits (ICs) of sufficient complexity use a clock signal in order to synchronize different parts of the circuit, cycling at a rate slower than the worst-case internal propagation delays. The preeminent example of the aforementioned integrated circuit is the CPU 320, the central component of modern computers, which relies on a clock. The only exceptions are asynchronous circuits such as asynchronous CPUs. The clock 310 can comprise a plurality of embodiments, such as, but not limited to, single-phase clock which transmits all clock signals on effectively 1 wire, two-phase clock which distributes clock signals on two wires, each with non-overlapping pulses, and four-phase clock which distributes clock signals on 4 wires.

Many computing devices 300 use a “clock multiplier” which multiplies a lower frequency external clock to the appropriate clock rate of the CPU 320. This allows the CPU 320 to operate at a much higher frequency than the rest of the computer, which affords performance gains in situations where the CPU 320 does not need to wait on an external factor (like memory 340 or input/output 360). Some embodiments of the clock 310 may include dynamic frequency change, where the time between clock edges can vary widely from one edge to the next and back again.

A system consistent with an embodiment of the disclosure the computing device 300 may include the CPU unit 320 comprising at least one CPU Core 321. A plurality of CPU cores 321 may comprise identical CPU cores 321, such as, but not limited to, homogeneous multi-core systems. It is also possible for the plurality of CPU cores 321 to comprise different CPU cores 321, such as, but not limited to, heterogeneous multi-core systems, big.LITTLE systems and some AMD accelerated processing units (APU). The CPU unit 320 reads and executes program instructions which may be used across many application domains, for example, but not limited to, general purpose computing, embedded computing, network computing, digital signal processing (DSP), and graphics processing (GPU). The CPU unit 320 may run multiple instructions on separate CPU cores 321 at the same time. The CPU unit 320 may be integrated into at least one of a single integrated circuit die and multiple dies in a single chip package. The single integrated circuit die and multiple dies in a single chip package may contain a plurality of other aspects of the computing device 300, for example, but not limited to, the clock 310, the CPU 320, the bus 330, the memory 340, and I/O 360.

The CPU unit 320 may contain cache 322 such as, but not limited to, a level 1 cache, level 2 cache, level 3 cache or combination thereof. The aforementioned cache 322 may or may not be shared amongst a plurality of CPU cores 321. The cache 322 sharing comprises at least one of message passing and inter-core communication methods may be used for the at least one CPU Core 321 to communicate with the cache 322. The inter-core communication methods may comprise, but not limited to, bus, ring, two-dimensional mesh, and crossbar. The aforementioned CPU unit 320 may employ symmetric multiprocessing (SMP) design.

The plurality of the aforementioned CPU cores 321 may comprise soft microprocessor cores on a single field programmable gate array (FPGA), such as semiconductor intellectual property cores (IP Core). The plurality of CPU cores 321 architecture may be based on at least one of, but not limited to, Complex instruction set computing (CISC), Zero instruction set computing (ZISC), and Reduced instruction set computing (RISC). At least one of the performance-enhancing methods may be employed by the plurality of the CPU cores 321, for example, but not limited to Instruction-level parallelism (ILP) such as, but not limited to, superscalar pipelining, and Thread-level parallelism (TLP).

Consistent with the embodiments of the present disclosure, the aforementioned computing device 300 may employ a communication system that transfers data between components inside the aforementioned computing device 300, and/or the plurality of computing devices 300. The aforementioned communication system will be known to a person having ordinary skill in the art as a bus 330. The bus 330 may embody internal and/or external plurality of hardware and software components, for example, but not limited to a wire, optical fiber, communication protocols, and any physical arrangement that provides the same logical function as a parallel electrical bus. The bus 330 may comprise at least one of, but not limited to a parallel bus, wherein the parallel bus carry data words in parallel on multiple wires, and a serial bus, wherein the serial bus carry data in bit-serial form. The bus 330 may embody a plurality of topologies, for example, but not limited to, a multidrop/electrical parallel topology, a daisy chain topology, and a connected by switched hubs, such as USB bus. The bus 330 may comprise a plurality of embodiments, for example, but not limited to:
Internal data bus (data bus) 331/Memory bus
Control bus 332
Address bus 333
System Management Bus (SMBus)
Front-Side-Bus (FSB)
External Bus Interface (EBI)
Local bus
Expansion bus
Lightning bus
Controller Area Network (CAN bus)
Camera Link
ExpressCard
Advanced Technology management Attachment (ATA), including embodiments and derivatives such as, but not limited to, Integrated Drive Electronics (IDE)/Enhanced IDE (EIDE), ATA Packet Interface (ATAPI), Ultra-Direct Memory Access (UDMA), Ultra ATA (UATA)/Parallel ATA (PATA)/Serial ATA (SATA), CompactFlash (CF) interface, Consumer Electronics ATA (CE-ATA)/Fiber Attached Technology Adapted (FATA), Advanced Host Controller Interface (AHCI), SATA Express (SATAe)/External SATA (eSATA), including the powered embodiment eSATAp/Mini-SATA (mSATA), and Next Generation Form Factor (NGFF)/M.2.
Small Computer System Interface (SCSI)/Serial Attached SCSI (SAS)
HyperTransport
InfiniBand
RapidIO
Mobile Industry Processor Interface (MIPI)
Coherent Processor Interface (CAPI)
Plug-n-play
1-Wire
Peripheral Component Interconnect (PCI), including embodiments such as, but not limited to, Accelerated Graphics Port (AGP), Peripheral Component Interconnect eXtended (PCI-X), Peripheral Component Interconnect Express (PCI-e) (e.g., PCI Express Mini Card, PCI Express M.2 [Mini PCIe v2], PCI Express External Cabling [ePCIe], and PCI Express OCuLink [Optical Copper{Cu} Link]), Express Card, AdvancedTCA, AMC, Universal IO, Thunderbolt/Mini DisplayPort, Mobile PCIe (M-PCIe), U.2, and Non-Volatile Memory Express (NVMe)/Non-Volatile Memory Host Controller Interface Specification (NVMHCIS).

Industry Standard Architecture (ISA), including embodiments such as, but not limited to Extended ISA (EISA), PC/XT-bus/PC/AT-bus/PC/104 bus (e.g., PC/104-Plus, PCI/104-Express, PCI/104, and PCI-104), and Low Pin Count (LPC).
Music Instrument Digital Interface (MIDI)
Universal Serial Bus (USB), including embodiments such as, but not limited to, Media Transfer Protocol (MTP)/Mobile High-Definition Link (MHL), Device Firmware Upgrade (DFU), wireless USB, InterChip USB, IEEE 1394 Interface/Firewire, Thunderbolt, and eXtensible Host Controller Interface (xHCI).

Consistent with the embodiments of the present disclosure, the aforementioned computing device 300 may employ hardware integrated circuits that store information for immediate use in the computing device 300, know to the person having ordinary skill in the art as primary storage or memory 340. The memory 340 operates at high speed, distinguishing it from the non-volatile storage sub-module 361, which may be referred to as secondary or tertiary storage, which provides slow-to-access information but offers higher capacities at lower cost. The contents contained in memory 340, may be transferred to secondary storage via techniques such as, but not limited to, virtual memory and swap. The memory 340 may be associated with addressable semiconductor memory, such as integrated circuits consisting of silicon-based transistors, used for example as primary storage but also other purposes in the computing device 300. The memory 340 may comprise a plurality of embodiments, such as, but not limited to volatile memory, non-volatile memory, and semi-volatile memory. It should be understood by a person having ordinary skill in the art that the ensuing are non-limiting examples of the aforementioned memory:
Volatile memory which requires power to maintain stored information, for example, but not limited to, Dynamic Random-Access Memory (DRAM) 341, Static Random-Access Memory (SRAM) 342, CPU Cache memory 325, Advanced Random-Access Memory (A-RAM), and other types of primary storage such as Random-Access Memory (RAM).
Non-volatile memory which can retain stored information even after power is removed, for example, but not limited to, Read-Only Memory (ROM) 343, Programmable ROM (PROM) 344, Erasable PROM (EPROM) 345, Electrically Erasable PROM (EEPROM) 346 (e.g., flash memory and Electrically Alterable PROM [EAPROM]), Mask ROM (MROM), One Time Programable (OTP) ROM/Write Once Read Many (WORM), Ferroelectric RAM (FeRAM), Parallel Random-Access Machine (PRAM), Split-Transfer Torque RAM (STT-RAM), Silicon Oxime Nitride Oxide Silicon (SONOS), Resistive RAM (RRAM), Nano RAM (NRAM), 3D XPoint, Domain-Wall Memory (DWM), and millipede memory.
Semi-volatile memory which may have some limited non-volatile duration after power is removed but loses data after said duration has passed. Semi-volatile memory provides high performance, durability, and other valuable characteristics typically associated with volatile memory, while providing some benefits of true non-volatile memory. The semi-volatile memory may comprise volatile and non-volatile memory and/or volatile memory with battery to provide power after power is removed. The semi-volatile memory may comprise, but not limited to spin-transfer torque RAM (STT-RAM).

Consistent with the embodiments of the present disclosure, the aforementioned computing device 300 may employ the communication system between an information processing system, such as the computing device 300, and the outside world, for example, but not limited to, human, environment, and another computing device 300. The aforementioned communication system will be known to a person having ordinary skill in the art as I/O 360. The I/O module 360 regulates a plurality of inputs and outputs with regard to the computing device 300, wherein the inputs are a plurality of signals and data received by the computing device 300, and the outputs are the plurality of signals and data sent from the computing device 300. The I/O module 360 interfaces a plurality of hardware, such as, but not limited to, non-volatile storage 361, communication devices 362, sensors 363, and peripherals 364. The plurality of hardware is used by the at least one of, but not limited to, human, environment, and another computing device 300 to communicate with the present computing device 300. The I/O module 360 may comprise a plurality of forms, for example, but not limited to channel I/O, port mapped I/O, asynchronous I/O, and Direct Memory Access (DMA).

Consistent with the embodiments of the present disclosure, the aforementioned computing device 300 may employ the non-volatile storage sub-module 361, which may be referred to by a person having ordinary skill in the art as one of secondary storage, external memory, tertiary storage, off-line storage, and auxiliary storage. The non-volatile storage sub-module 361 may not be accessed directly by the CPU 320 without using intermediate area in the memory 340. The non-volatile storage sub-module 361 does not lose data when power is removed and may be two orders of magnitude less costly than storage used in memory module, at the expense of speed and latency. The non-volatile storage sub-module 361 may comprise a plurality of forms, such as, but not limited to, Direct Attached Storage (DAS), Network Attached Storage (NAS), Storage Area Network (SAN), nearline storage, Massive Array of Idle Disks (MAID), Redundant Array of Independent Disks (RAID), device mirroring, off-line storage, and robotic storage. The non-volatile storage sub-module (361) may comprise a plurality of embodiments, such as, but not limited to:
Optical storage, for example, but not limited to, Compact Disk (CD) (CD-ROM/CD-R/CD-RW), Digital Versatile Disk (DVD) (DVD-ROM/DVD-R/DVD+R/DVD-RW/DVD+RW/DVD±RW/DVD+R DL/DVD-RAM/HD-DVD), Blu-ray Disk (BD) (BD-ROM/BD-R/BD-RE/BD-R DL/BD-RE DL), and Ultra-Density Optical (UDO).
Semiconductor storage, for example, but not limited to, flash memory, such as, but not limited to, USB flash drive, Memory card, Subscriber Identity Module (SIM) card, Secure Digital (SD) card, Smart Card, CompactFlash (CF) card, Solid-State Drive (SSD) and memristor.
Magnetic storage such as, but not limited to, Hard Disk Drive (HDD), tape drive, carousel memory, and Card Random-Access Memory (CRAM).
Phase-change memory
Holographic data storage such as Holographic Versatile Disk (HVD).
Molecular Memory
Deoxyribonucleic Acid (DNA) digital data storage

Consistent with the embodiments of the present disclosure, the aforementioned computing device 300 may employ the communication sub-module 362 as a subset of the I/O 360, which may be referred to by a person having ordinary skill in the art as at least one of, but not limited to, computer network, data network, and network. The network allows computing devices 300 to exchange data using connections, which may be known to a person having ordinary skill in the art as data links, between network nodes. The nodes comprise network computer devices 300 that originate, route, and terminate data. The nodes are identified by network addresses and can include a plurality of hosts consistent with the embodiments of a computing device 300. The aforementioned embodiments include, but not limited to personal computers, phones, servers, drones, and networking devices such as, but not limited to, hubs, switches, routers, modems, and firewalls.

Two nodes can be said are networked together, when one computing device 300 is able to exchange information with the other computing device 300, whether or not they have a direct connection with each other. The communication sub-module 362 supports a plurality of applications and services, such as, but not limited to World Wide Web (WWW), digital video and audio, shared use of application and storage computing devices 300, printers/scanners/fax machines, email/online chat/instant messaging, remote control, distributed computing, etc. The network may comprise a plurality of transmission mediums, such as, but not limited to conductive wire, fiber optics, and wireless. The network may comprise a plurality of communications protocols to organize network traffic, wherein application-specific communications protocols are layered, may be known to a person having ordinary skill in the art as carried as payload, over other more general communications protocols. The plurality of communications protocols may comprise, but not limited to, IEEE 802, ethernet, Wireless LAN (WLAN/Wi-Fi), Internet Protocol (IP) suite (e.g., TCP/IP, UDP, Internet Protocol version 4 [IPv4], and Internet Protocol version 6 [IPv6]), Synchronous Optical Networking (SONET)/Synchronous Digital Hierarchy (SDH), Asynchronous Transfer Mode (ATM), and cellular standards (e.g., Global System for Mobile Communications [GSM], General Packet Radio Service [GPRS], Code-Division Multiple Access [CDMA], and Integrated Digital Enhanced Network [IDEN]).

The communication sub-module 362 may comprise a plurality of size, topology, traffic control mechanism and organizational intent. The communication sub-module 362 may comprise a plurality of embodiments, such as, but not limited to:
Wired communications, such as, but not limited to, coaxial cable, phone lines, twisted pair cables (ethernet), and InfiniBand.
Wireless communications, such as, but not limited to, communications satellites, cellular systems, radio frequency/spread spectrum technologies, IEEE 802.11 Wi-Fi, Bluetooth, NFC, free-space optical communications, terrestrial microwave, and Infrared (IR) communications. Wherein cellular systems embody technologies such as, but not limited to, 3G, 4G (such as WiMax and LTE), and 5G (short and long wavelength).
Parallel communications, such as, but not limited to, LPT ports.
Serial communications, such as, but not limited to, RS-232 and USB.
Fiber Optic communications, such as, but not limited to, Single-mode optical fiber (SMF) and Multi-mode optical fiber (MMF).
Power Line communications

The aforementioned network may comprise a plurality of layouts, such as, but not limited to, bus network such as ethernet, star network such as Wi-Fi, ring network, mesh network, fully connected network, and tree network. The network can be characterized by its physical capacity or its organizational purpose. Use of the network, including user authorization and access rights, differ accordingly. The characterization may include, but not limited to nanoscale network, Personal Area Network (PAN), Local Area Network (LAN), Home Area Network (HAN), Storage Area Network (SAN), Campus Area Network (CAN), backbone network, Metropolitan Area Network (MAN), Wide Area Network (WAN), enterprise private network, Virtual Private Network (VPN), and Global Area Network (GAN).

Consistent with the embodiments of the present disclosure, the aforementioned computing device 300 may employ the sensors sub-module 363 as a subset of the I/O 360. The sensors sub-module 363 comprises at least one of the devices, modules, and subsystems whose purpose is to detect events or changes in its environment and send the information to the computing device 300. Sensors are sensitive to the measured property, are not sensitive to any property not measured, but may be encountered in its application, and do not significantly influence the measured property. The sensors sub-module 363 may comprise a plurality of digital devices and analog devices, wherein if an analog device is used, an Analog to Digital (A-to-D) converter must be employed to interface the said device with the computing device 300. The sensors may be subject to a plurality of deviations that limit sensor accuracy. The sensors sub-module 363 may comprise a plurality of embodiments, such as, but not limited to, chemical sensors, automotive sensors, acoustic/sound/vibration sensors, electric current/electric potential/magnetic/radio sensors, environmental/weather/moisture/humidity sensors, flow/fluid velocity sensors, ionizing radiation/particle sensors, navigation sensors, position/angle/displacement/distance/speed/acceleration sensors, imaging/optical/light sensors, pressure sensors, force/density/level sensors, thermal/temperature sensors, and proximity/presence sensors. It should be understood by a person having ordinary skill in the art that the ensuing are non-limiting examples of the aforementioned sensors:
Chemical sensors, such as, but not limited to, breathalyzer, carbon dioxide sensor, carbon monoxide/smoke detector, catalytic bead sensor, chemical field-effect transistor, chemiresistor, electrochemical gas sensor, electronic nose, electrolyte-insulator-semiconductor sensor, energy-dispersive X-ray spectroscopy, fluorescent chloride sensors, holographic sensor, hydrocarbon dew point analyzer, hydrogen sensor, hydrogen sulfide sensor, infrared point sensor, ion-selective electrode, nondispersive infrared sensor, microwave chemistry sensor, nitrogen oxide sensor, olfactometer, optode, oxygen sensor, ozone monitor, pellistor, pH glass electrode, potentiometric sensor, redox electrode, zinc oxide nanorod sensor, and biosensors (such as nanosensors).
Automotive sensors, such as, but not limited to, air flow meter/mass airflow sensor, air-fuel ratio meter, AFR sensor, blind spot monitor, engine coolant/exhaust gas/cylinder head/transmission fluid temperature sensor, hall effect sensor, wheel/automatic transmission/turbine/vehicle speed sensor, airbag sensors, brake fluid/engine crankcase/fuel/oil/tire pressure sensor, camshaft/crankshaft/throttle position sensor, fuel/oil level sensor, knock sensor, light sensor, MAP sensor, oxygen sensor (o2), parking sensor, radar sensor, torque sensor, variable reluctance sensor, and water-in-fuel sensor.
Acoustic, sound and vibration sensors, such as, but not limited to, microphone, lace sensor (guitar pickup), seismometer, sound locator, geophone, and hydrophone.
Electric current, electric potential, magnetic, and radio sensors, such as, but not limited to, current sensor, Daly detector, electroscope, electron multiplier, faraday cup, galvanometer, hall effect sensor, hall probe, magnetic anomaly detector, magnetometer, magnetoresistance, MEMS magnetic field sensor, metal detector, planar hall sensor, radio direction finder, and voltage detector.
Environmental, weather, moisture, and humidity sensors, such as, but not limited to, actinometer, air pollution sensor, bedwetting alarm, ceilometer, dew warning, electrochemical gas sensor, fish counter, frequency domain sensor, gas detector, hook gauge evaporimeter, humistor, hygrometer, leaf sensor, lysimeter, pyranometer, pyrgeometer, psychrometer, rain gauge, rain sensor, seismometers, SNOTEL, snow gauge, soil moisture sensor, stream gauge, and tide gauge.
Flow and fluid velocity sensors, such as, but not limited to, air flow meter, anemometer, flow sensor, gas meter, mass flow sensor, and water meter.
Ionizing radiation and particle sensors, such as, but not limited to, cloud chamber, Geiger counter, Geiger-Muller tube, ionization chamber, neutron detection, proportional counter, scintillation counter, semiconductor detector, and thermoluminescent dosimeter.
Navigation sensors, such as, but not limited to, air speed indicator, altimeter, attitude indicator, depth gauge, fluxgate compass, gyroscope, inertial navigation system, inertial reference unit, magnetic compass, MHD sensor, ring laser gyroscope, turn coordinator, variometer, vibrating structure gyroscope, and yaw rate sensor.
Position, angle, displacement, distance, speed, and acceleration sensors, such as, but not limited to, accelerometer, displacement sensor, flex sensor, free fall sensor, gravimeter, impact sensor, laser rangefinder, LIDAR, odometer, photoelectric sensor, position sensor such as, but not limited to, GPS or Glonass, angular rate sensor, shock detector, ultrasonic sensor, tilt sensor, tachometer, ultra-wideband radar, variable reluctance sensor, and velocity receiver.
Imaging, optical and light sensors, such as, but not limited to, CMOS sensor, colorimeter, contact image sensor, electro-optical sensor, infra-red sensor, kinetic inductance detector, LED as light sensor, light-addressable potentiometric sensor, Nichols radiometer, fiber-optic sensors, optical position sensor, thermopile laser sensor, photodetector, photodiode, photomultiplier tubes, phototransistor, photoelectric sensor, photoionization detector, photomultiplier, photoresistor, photoswitch, phototube, scintillometer, Shack-Hartmann, single-photon avalanche diode, superconducting nanowire single-photon detector, transition edge sensor, visible light photon counter, and wavefront sensor.
Pressure sensors, such as, but not limited to, barograph, barometer, boost gauge, bourdon gauge, hot filament ionization gauge, ionization gauge, McLeod gauge, Oscillating U-tube, permanent downhole gauge, piezometer, Pirani gauge, pressure sensor, pressure gauge, tactile sensor, and time pressure gauge.
Force, Density, and Level sensors, such as, but not limited to, bhangmeter, hydrometer, force gauge or force sensor, level sensor, load cell, magnetic level or nuclear density sensor or strain gauge, piezocapacitive pressure sensor, piezoelectric sensor, torque sensor, and viscometer.
Thermal and temperature sensors, such as, but not limited to, bolometer, bimetallic strip, calorimeter, exhaust gas temperature gauge, flame detection/pyrometer, Gardon gauge, Golay cell, heat flux sensor, microbolometer, microwave radiometer, net radiometer, infrared/quartz/resistance thermometer, silicon bandgap temperature sensor, thermistor, and thermocouple.
Proximity and presence sensors, such as, but not limited to, alarm sensor, doppler radar, motion detector, occupancy sensor, proximity sensor, passive infrared sensor, reed switch, stud finder, triangulation sensor, touch switch, and wired glove.

Consistent with the embodiments of the present disclosure, the aforementioned computing device 300 may employ the peripherals sub-module 362 as a subset of the I/O 360. The peripheral sub-module 364 comprises ancillary devices uses to put information into and get information out of the computing device 300. There are 3 categories of devices comprising the peripheral sub-module 364, which exist based on their relationship with the computing device 300, input devices, output devices, and input/output devices. Input devices send at least one of data and instructions to the computing device 300. Input devices can be categorized based on, but not limited to:
Modality of input, such as, but not limited to, mechanical motion, audio, visual, and tactile.
Whether the input is discrete, such as but not limited to, pressing a key, or continuous such as, but not limited to position of a mouse.
The number of degrees of freedom involved, such as, but not limited to, two-dimensional mice vs three-dimensional mice used for Computer-Aided Design (CAD) applications.

Output devices provide output from the computing device 300. Output devices convert electronically generated information into a form that can be presented to humans. Input/output devices perform that perform both input and output functions. It should be understood by a person having ordinary skill in the art that the ensuing are non-limiting embodiments of the aforementioned peripheral sub-module 364:
Input Devices
   Human Interface Devices (HID), such as, but not limited to, pointing device (e.g., mouse, touchpad, joystick, touchscreen, game controller/gamepad, remote, light pen, light gun, Wii remote, jog dial, shuttle, and knob), keyboard, graphics tablet, digital pen, gesture recognition devices, magnetic ink character recognition, Sip-and-Puff (SNP) device, and Language Acquisition Device (LAD).
   High degree of freedom devices, that require up to six degrees of freedom such as, but not limited to, camera gimbals, Cave Automatic Virtual Environment (CAVE), and virtual reality systems.
   Video Input devices are used to digitize images or video from the outside world into the computing device 300. The information can be stored in a multitude of formats depending on the user's requirement. Examples of types of video input devices include, but not limited to, digital camera, digital camcorder, portable media player, webcam, Microsoft Kinect, image scanner, fingerprint scanner, barcode reader, 3D scanner, laser rangefinder, eye gaze tracker, computed tomography, magnetic resonance imaging, positron emission tomography, medical ultrasonography, TV tuner, and iris scanner.
   Audio input devices are used to capture sound. In some cases, an audio output device can be used as an input device, in order to capture produced sound. Audio input devices allow a user to send audio signals to the computing device 300 for at least one of processing, recording, and carrying out commands. Devices such as microphones allow users to speak to the computer in order to record a voice message or navigate software. Aside from recording, audio input devices are also used with speech recognition software. Examples of types of audio input devices include, but not limited to microphone, Musical Instrumental Digital Interface (MIDI) devices such as, but not limited to a keyboard, and headset.
   Data AcQuisition (DAQ) devices convert at least one of analog signals and physical parameters to digital values for processing by the computing device 300. Examples of DAQ devices may include, but not limited to, Analog to Digital Converter (ADC), data logger, signal conditioning circuitry, multiplexer, and Time to Digital Converter (TDC).
Output Devices may further comprise, but not be limited to:
   Display devices, which convert electrical information into visual form, such as, but not limited to, monitor, TV, projector, and Computer Output Microfilm (COM). Display devices can use a plurality of underlying technologies, such as, but not limited to, Cathode-Ray Tube (CRT), Thin-Film Transistor (TFT), Liquid Crystal Display (LCD), Organic Light-Emitting Diode (OLED), MicroLED, E Ink Display (ePaper) and Refreshable Braille Display (Braille Terminal).
   Printers, such as, but not limited to, inkjet printers, laser printers, 3D printers, solid ink printers and plotters.
   Audio and Video (AV) devices, such as, but not limited to, speakers, headphones, amplifiers and lights, which include lamps, strobes, DJ lighting, stage lighting, architectural lighting, special effect lighting, and lasers.
   Other devices such as Digital to Analog Converter (DAC)
Input/Output Devices may further comprise, but not be limited to, touchscreens, networking device (e.g., devices disclosed in network 362 sub-module), data storage device (non-volatile storage 361), facsimile (FAX), and graphics/sound cards.

All rights including copyrights in the code included herein are vested in and the property of the Applicant. The Applicant retains and reserves all rights in the code included herein, and grants permission to reproduce the material only in connection with reproduction of the granted patent and for no other purpose.

### V. Claims

While the specification includes examples, the disclosure's scope is indicated by the following claims. Furthermore, while the specification has been described in language specific to structural features and/or methodological acts, the claims are not limited to the features or acts described above. Rather, the specific features and acts described above are disclosed as examples for embodiments of the disclosure.

Insofar as the description above and the accompanying drawing disclose any additional subject matter that is not within the scope of the claims below, the disclosures are not dedicated to the public and the right to file one or more applications to claims such additional disclosures is reserved.

The following is claimed:

1. One or more non-transitory computer readable media comprising instructions which, when executed by one or more hardware processors, causes performance of operations comprising:
receiving, from a health care provider, an order comprising patient identification information and an indication of a proposed drug to be administered to the patient;
gathering patient information based on the patient identification information, the patient information comprising a diagnostic history and a list of prescribed medications;
testing a bodily fluid sample from a patient, the bodily fluid sample test determining one or more of:
one or more substances present within the patient, or
one or more patient conditions;
determining one or more contraindications associated with the proposed drug, the one or more contraindications being based on one or more of:
a diagnosis present in the diagnostic history,
one of the one or more substances present within the patient, or
a condition determined by the bodily fluid test; and
providing a report to a health care provider, the report including the one or more determined contraindications.

2. The one or more non-transitory computer readable media of claim 1, wherein testing a bodily fluid sample comprises:
receiving a bodily fluid sample from the patient;
performing a gas chromatography mass spectrometry analysis of the fluid sample;
determining, based on the gas chromatography mass spectrometry analysis of the fluid sample, one or more chemicals in the fluid;
generating a list of substances taken by the patient based on the determined chemicals in the fluid;
calculating, for each substance in the list of substances, an associated confidence level indicating a likelihood that the patient has taken the substance; and
storing a list of the substances together with their associated confidence levels.

3. The one or more non-transitory computer readable media of claim 2, wherein determining the one or more contraindications comprises:
transmitting, to an interaction database, a query comprising:
the proposed drug, and
one or more substances included in the list of substances; and
receiving, from the interaction database, an indication of one or more drug-drug interactions based on the proposed drug and the one or more substances included in the list of substances.

4. The one or more non-transitory computer readable media of claim 3, wherein the one or more substances included in the list of substances comprises all substance in the list of substance having an associated confidence level that exceeds a predetermined threshold.

5. one or more non-transitory computer readable media of claim 2, wherein determining the one or more contraindications comprises:
retrieving one or more patient medical conditions from an Electronic Medical Record (EMR) based on the patient identification information of the order;
transmitting to an interaction database, a query comprising:
the proposed drug, and
the one or more patient medical conditions; and
receiving, from the interaction database, an indication of one or more contraindications based on the proposed drug and the one or more patient medical conditions.

6. The one or more non-transitory computer readable media of claim 1, wherein providing a report to a health care provider comprises storing the report to an EMR associated with the patient based on the patient identification information of the order, wherein the health care provider has access to the EMR.

7. The one or more non-transitory computer readable media of claim 1, wherein the bodily fluid comprises one or more of:
blood,
urine,
cerebrospinal fluid,
saliva, or
sweat.

8. A method comprising:
receiving, from a health care provider, an order comprising patient identification information and an indication of a proposed drug to be administered to the patient;
gathering patient information based on the patient identification information, the patient information comprising a diagnostic history and a list of prescribed medications;
testing a bodily fluid sample from a patient, the bodily fluid sample test determining one or more of:
one or more substances present within the patient, or
one or more patient conditions;
determining one or more contraindications associated with the proposed drug, the one or more contraindications being based on one or more of:
a diagnosis present in the diagnostic history,
one of the one or more substances present within the patient, or
a condition determined by the bodily fluid test; and
providing a report to a health care provider, the report including the one or more determined contraindications.

9. The method of claim 8, wherein testing a bodily fluid sample comprises:
receiving a bodily fluid sample from the patient;
performing a gas chromatography mass spectrometry analysis of the fluid sample;
determining, based on the gas chromatography mass spectrometry analysis of the fluid sample, one or more chemicals in the fluid;
generating a list of substances taken by the patient based on the determined chemicals in the fluid;
calculating, for each substance in the list of substances, an associated confidence level indicating a likelihood that the patient has taken the substance; and
storing a list of the substances together with their associated confidence levels.

10. The method of claim 9, wherein determining the one or more contraindications comprises:
transmitting, to an interaction database, a query comprising:
the proposed drug, and
one or more substances included in the list of substances; and
receiving, from the interaction database, an indication of one or more drug-drug interactions based on the proposed drug and the one or more substances included in the list of substances.

11. The method of claim 10, wherein the one or more substances included in the list of substances comprises all substance in the list of substance having an associated confidence level that exceeds a predetermined threshold.

12. The method of claim 9, wherein determining the one or more contraindications comprises:
retrieving one or more patient medical conditions from an Electronic Medical Record (EMR) based on the patient identification information of the order;
transmitting to an interaction database, a query comprising:
the proposed drug, and
the one or more patient medical conditions; and
receiving, from the interaction database, an indication of one or more contraindications based on the proposed drug and the one or more patient medical conditions.

13. The method of claim 8, wherein providing a report to a health care provider comprises storing the report to an EMR associated with the patient based on the patient identification information of the order, wherein the health care provider has access to the EMR.

14. The method of claim 8, wherein the bodily fluid comprises one or more of:
blood,
urine,
cerebrospinal fluid,
saliva, or
sweat.

15. A system comprising:
at least one device including a hardware processor;
the system being configured to perform operations comprising:
receiving, from a health care provider, an order comprising patient identification information and an indication of a proposed drug to be administered to the patient;
gathering patient information based on the patient identification information, the patient information comprising a diagnostic history and a list of prescribed medications;
testing a bodily fluid sample from a patient, the bodily fluid sample test determining one or more of:
one or more substances present within the patient, or
one or more patient conditions;
determining one or more contraindications associated with the proposed drug, the one or more contraindications being based on one or more of:
a diagnosis present in the diagnostic history,
one of the one or more substances present within the patient, or
a condition determined by the bodily fluid test; and
providing a report to a health care provider, the report including the one or more determined contraindications.

16. The system of claim 15, wherein testing a bodily fluid sample comprises:
receiving a bodily fluid sample from the patient;
performing a gas chromatography mass spectrometry analysis of the fluid sample;
determining, based on the gas chromatography mass spectrometry analysis of the fluid sample, one or more chemicals in the fluid;
generating a list of substances taken by the patient based on the determined chemicals in the fluid;
calculating, for each substance in the list of substances, an associated confidence level indicating a likelihood that the patient has taken the substance; and
storing a list of the substances together with their associated confidence levels.

17. The system of claim 16, wherein determining the one or more contraindications comprises:
transmitting, to an interaction database, a query comprising:
the proposed drug, and
one or more substances included in the list of substances; and
receiving, from the interaction database, an indication of one or more drug-drug interactions based on the proposed drug and the one or more substances included in the list of substances.

18. The system of claim 17, wherein the one or more substances included in the list of substances comprises all substance in the list of substance having an associated confidence level that exceeds a predetermined threshold.

19. The system of claim 16, wherein determining the one or more contraindications comprises:
retrieving one or more patient medical conditions from an Electronic Medical Record (EMR) based on the patient identification information of the order;
transmitting to an interaction database, a query comprising:
the proposed drug, and
the one or more patient medical conditions; and
receiving, from the interaction database, an indication of one or more contraindications based on the proposed drug and the one or more patient medical conditions.

20. The system of claim 15, wherein providing a report to a health care provider comprises storing the report to an EMR associated with the patient based on the patient identification information of the order, wherein the health care provider has access to the EMR.
